(19) United States
(12) Patent Application Publication
(10) Pub. No.: US 2004/0023962 A1
(43) Pub. Date: Feb. 5, 2004

(54) Use of medicaments

(76) Inventor: Steven Etheridge, Harlow, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/398,416

(22) PCT Filed: Oct. 5, 2001
(86) PCT No.: PCT/EP01/11498

(30) Foreign Application Priority Data
Oct. 6, 2000 (GB) 0024517.5

(51) Int. Cl. A61K 31/53

(52) U.S. Cl. 514/242

(57) ABSTRACT

The present invention provides the use of frequency dependent voltage activated sodium channel blockers particularly lamotrigine in the prevention of noise induced hearing loss.

The present invention relates to a new use of frequency dependent voltage activated sodium channel blockers. In particular, the present invention relates to a new use of 3,5-diamino-6-(2,3-dichlorophenyl)-1,2,4-triazine and its pharmaceutically acceptable acid addition salts.

Compounds exhibiting frequency dependent voltage activated sodium channel blocker activity include those described in EP-A-0021121, WO97/09317, WO98/38174, WO99/32462 and WO00/12488.

Simpson et al., The Assessment of Lamotrigine, an Antieplileptic Drug, in The Treatment of Tinnitus, Am J Otol 1999; 20: p 627-631 suggests that lamotrigine might be useful in the treatment of tinnitus.

NIHL is the most frequent occupational disease with prevalence of 30 to 50% in exposed populations. At present the only effective method for preventing NIHL is for the subject to wear some form of physical shield, such as ear protectors. However, ear protectors are only of use when the subject is aware the he is likely to be exposed to a noise insult and in some circumstances, such as in a battlefield situation, exposure to a noise insult can be sudden and unexpected. Moreover, the use of ear protectors affects the subjects hearing across its whole range often making quiet noises, such as conversation, inaudible. Accordingly, there is a need for alternative means for protecting the ear against noise induced hearing loss which does not affect normal auditory function. In particular, there is a need for protecting the ear against hearing loss resulting from acute or chronic noise induced hearing loss which does not affect normal auditory function.

It has now been surprisingly found that frequency dependent voltage activated sodium channel blockers, particularly 3,5-diamino-6-(2,3-dichlorophenyl)-1,2,4-triazine and its pharmaceutically acceptable acid addition salts, are effective in the prevention of noise induced hearing loss (NIHL).

Accordingly, the present invention provides the use of a frequency dependent voltage activated sodium channel blocker or a pharmaceutically acceptable acid addition salt thereof in the manufacture of a medicament for the prevention of noise induced hearing loss (NIHL).

### BRIEF DESCRIPTION OF THE FIGURES

FIG. 1 illustrates the pharmacokinetic profile of 3,5-diamino-6-(2,3-dichlorophenyl)-1,2,4-triazine in plasma and perilymph.

FIG. 2 demonstrates the effect of 3,5-diamino-6-(2,3-dichlorophenyl)-1,2,4-triazine on cochlea nerve compound action potential threshold shifts in the absence of auditory stimulation.

FIG. 3 illustrates the effect 3,5-diamino-6-(2,3-dichlorophenyl)-1,2,4-triazine on cochlea nerve compound action potential threshold shifts in response to discrete acoustic stimuli (110 dB SPL).

FIG. 4 demonstrates the effect of 3,5-diamino-6-(2,3-dichlorophenyl)-1,2,4-triazine on the gross ensemble spontaneous activity of the cochlea nerve fibre before during and after noise exposure (110 dB spl).

3,5-Diamino-6-(2,3-dichlorophenyl)-1,2,4-triazine will herein after be referred to as lamotrigine.

The term prevention as used herein with reference to prevention of noise induced hearing loss (NIHL) means to preclude, or ameliorate the progression of hearing loss.

The term noise induced hearing loss (NIHL) as used herein is intended to refer to the loss of hearing performance, marked by a loss of sensitivity and resolution in hearing resulting from over-stimulation of the auditory system. In particular, the term NIHL is intended to refer to the loss in hearing performance resulting from damage to the cochlear nerve as a result of prolonged or acute exposure to noise stimuli.

It is thought that voltage activated sodium channel blockers act by interacting with voltage activated sodium channels in such a way as to reduce the flow of sodium current when these channels are open. The term frequency dependent as used herein with reference to voltage activated sodium channel blockers is intended to cover those compounds whose interaction with the sodium channels is dependent on the rate the channels open and subsequently inactivate. Thus the effect of frequency dependent voltage activated sodium channel blockers will vary depending on the frequency with which the sodium channels open and inactivate.

Determination of the frequency dependence of a voltage activated sodium channel blocker requires sodium channels to be repetitively activated at a range of frequencies, in the presence and absence of the blocker. This may be achieved by applying depolarising pulses to elicit sodium currents using the whole-cell voltage-clamp technique and recording from a cell type containing voltage-activated sodium channels, for example Chinese hamster ovary cells expressing recombinant human brain type IIA sodium channels. As the frequency of the depolarisations is increased the degree of blockade increases, i.e. frequency dependence. Conversely, non-frequency dependent blockers will produce the same level of blockade for the complete range of frequencies applied. For a more detailed description of determination of frequency dependence see Ragsdale D S, Scheuer T, Catterall W A. Frequency and voltage-dependent inhibition of type IIA Na+ channels, expressed in a mammalian cell line, by local anesthetic, antiarrhythmic, and anticonvulsant drugs. Mol Pharmacol November 1991;40(5):756-65.

Suitable examples of frequency dependent voltage activated sodium channel blockers include those described in EP-A-0021121, WO97/09317, WO98/38174, WO99/32462 and WO0/12488 all incorporated herein by reference. A suitable compound described in EP-A-0021121 is lamotrigine and pharmaceutically acceptable acid addition salts thereof. A suitable compound described in WO97/09317 is R(−)-2,4-diamino-5-(2,3-dichlorophenyl)6-fluoromethyl pyrimidine and pharmaceutically acceptable acid addition salts thereof. A suitable compound described in WO98/38174 is 3-(2,3,5-trichloro-phenyl)-pyrazine-2,6-diamine and pharmaceutically acceptable acid addition salts thereof. A suitable compound described in WO99/32462 is 5-amino-6-[2,3,5-trichlorophenyl]-1,2,4-triazine and pharmaceutically acceptable acid addition salts thereof. A suitable compound described in WO00/12488 is 2,6-diamino-5-carboxamido-3-(2,3,5-trichlorophenyl)pyrazine and pharmaceutically acceptable acid addition salts thereof. A preferred frequency dependent voltage activated sodium channel blocker is lamotrigine and its pharmaceutically acceptable acid addition salts.

Suitable pharmaceutically acceptable acid addition salts include those formed with both organic and inorganic acids. Examples of such salts include those formed with hydrochloric, sulphuric, citric, tartaric, phosphoric, lactic, pyruvic, acetic, succinic, fumaric, maleic, methanesulphonic, ethanesulphonic, oxaloacetic and isethionic acids.

As reported in the Examples that follow the otoprotective effect of the frequency dependent voltage activated sodium channel blocker, lamotrigine, against acute noise exposure was determined in the guinea pig model.

The experimental results presented herein demonstrate penetration of the guinea pig cochlea by lamotrigine. The absence of an effect of lamotrigine on the normal sensitivity of the cochlea over 8-30 kHz together with the absence of an effect on the normal gross spontaneous activity of the cochlear nerve, as measured by analysis of cochlear spontaneous noise signal, demonstrates that in the absence of traumatic noise lamotrigine has no discernible effect on normal auditory function.

The results further demonstrate that the elevation in threshold (the level of sound that was required to generate the smallest detectable electrophysiological response) caused by acute exposure to noise can be ameliorated if lamotrigine is administered prior to noise exposure. Moreover, the results establish that administration of lamotrigine prior to noise exposure can reduce the total amount of driven activity in the cochlear nerve. Suitably, the total amount of driven activity in the cochlear nerve is reduced by more than 30%, preferably by more than 50%.

Without wishing to be bound by theory, it is believed that lamotrigine acts at the voltage activated sodium channels in the cochlear nerve by reducing the number of channels in the open state in a frequency dependent manner. It is thought to do achieve this by stabilising and prolonging the inactivation state of these channels. This reduces over stimulation of cochlear nerve fibres and thus prevents or ameliorates excitotoxic damage to these nerve fibres.

Accordingly, the present invention further provides a method of preventing noise induced hearing loss in a patient suffering from, or susceptible to, said disorder, which method comprises administering to the patient a therapeutically effective amount of a frequency dependent voltage activated sodium channel blocker or pharmaceutically acceptable acid addition salts thereof.

Compounds for use in the invention may be administered at a dose of from 0.1 to 40 mg/kg body weight per day, suitably 0.3 to 30 mg/kg body weight per day and more particularly 1 to 10 mg/kg weight per day, calculated as the free base. The dose range for adult human beings is generally from 8 to 2000 mg/day, such as from 35 to 1200 mg/day, preferably 10 to 500 mg/day or 20 to 200 mg/day, calculated as the free base. A particularly suitable dose range for adult human beings for lamotrigine is 100-400 mg/day.

While it is possible for the compounds to be administered as the raw chemical, it is preferable to present it as a pharmaceutical formulation. The formulations of the present invention comprise a frequency dependent voltage activated sodium channel blocker, such as lamotrigine and its pharmaceutically acceptable acid addition salts thereof, together with one or more acceptable carriers or diluents therefor and optionally other therapeutic ingredients. The carrier(s) must be “acceptable” in the sense of being compatible with the other ingredients of the formulation and not deleterious to the recipient thereof.

The formulations include those suitable for oral, parenteral (including subcutaneous e.g. by injection or by depot tablet, intradermal, intrathecal, intramuscular e.g. by depot and intravenous), rectal and topical (including dermal, buccal and sublingual) administration and intracochlear perfusion/infusion although the most suitable route may depend upon for example the condition of the recipient. The formulations may conveniently be presented in unit dosage form and may be prepared by any of the methods well known in the art of pharmacy. All methods include the step of bringing into association the compounds (“active ingredient”) with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of a sterile liquid carrier, for example, water-for-injection, immediately prior to use. Extemporaneous injection solutions and suspensions may be prepared from sterile powders, granules and tablets of the kind previously described.

Formulations for rectal administration may be presented as a suppository with the usual carriers such as cocoa butter, hard fat or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

In addition to the ingredients particularly mentioned above, the formulations may include other agents conventional in the art having regard to the type of formulation in question, for example those suitable for oral administration may include flavouring agents.

The compounds may be used in combination with other therapeutic agents, for example other frequency dependent voltage activated sodium channel blockers. When compounds are used in combination with other therapeutic agents, the compounds may be administered either sequentially or simultaneously by any convenient route. The invention thus provides, in a further aspect, a combination comprising a frequency dependent voltage activated sodium channel blocker or a pharmaceutically acceptable derivative thereof with a further therapeutic agent for the prevention of noise induced hearing loss.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

R(−)-2,4-diamino-5-(2,3-dichlorophenyl)-6-fluoromethyl pyrimidine may be prepared according to the Examples described in WO97/09317. 3-(2,3,5-trichloro-phenyl)-pyrazine-2,6-diamine may be prepared according to the methods described in WO98/38174, see particularly Example 1. 5-amino-6-[2,3,5-trichlorophenyl]-1,2,4-triazine may be prepared according to the methods described in WO99/32462, see particularly the Example. 2,6-diamino-5-carboxamido-3-(2,3,5-trichlorophenyl)pyrazine may be prepared according to the methods described in WO00/12488, see particularly Example 1.

Lamotrigine may be prepared by a process which comprises cyclising the compound of formula (I):

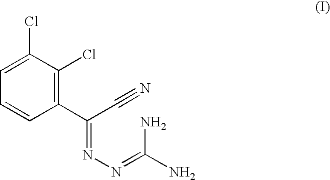

and, if desired, converting lamotrigine thus obtained into a pharmaceutically acceptable acid addition salt.

The cyclisation is typically carried out by heating the compound of formula (I) under reflux in an alkanol, preferably a C₁₋₄ alkanol, for example methanol or ethanol, in the presence of a strong base, for example potassium hydroxide.

The process may, for instance, be carried out as described in Example 1 of EP-A-0021121. The optional subsequent step of converting the lamotrigine into an acid addition salt is performed by a conventional method, for example by treatment with the appropriate acid at ambient temperature. The salt with isethionic acid may be prepared, for instance, as described in EP-A-0 247 892, in particular in Example 3. The starting compound of formula (I) may be prepared by the method described in U.S. Pat. No. 3,637,688.

The invention is further illustrated in the Examples which follow.

### BIOLOGICAL EXAMPLES

Experimental Aim

The aim of these studies was to establish whether lamotrigine (LTG) would have any otoprotective effect against acute noise exposure in a guinea pig model.

Justification of LTG Dose Level

Data in the rat has shown an effective oral dose (ED₅₀) of 6.8 mg/kg in the supramaximal electroshock (MES) model. Based on this data and pharmacokinetic data in the guinea pig (Parsons et al, 1995) a single oral dose of 20 mg/kg (4 mg/mL) in sterile water for irrigation was administered. All solutions were prepared fresh on the day of dosing and stored at 2-8° C., protected from light, between dosing sessions.

Surgical Procedure

Sedation was achieved using an intramuscular injection of 0.5 mL/kg medetomidine followed by an intraperitoneal injection of 6 mL/kg fentanyl approximately 10 minutes later (loss of the pedal reflex was used to determine sufficient sedation for surgery). Supplementary doses of both agents were administered as necessary during the experiment.

The animal was placed in a prone position (left ear upwards) in a purpose built perspex chamber for the duration of the experiment. Core body temperature was maintained at 37° C.+/−1° C. and a non-invasive pulse oximeter was placed on the forepaw to measure blood oxygenation and heart rate. Both were kept within normal limits for the guinea pig throughout each experiment.

A combination recording electrode and drug delivery catheter was placed through the bulla into the round window niche with the aid of a surgical microscope. A cotton wick was placed around the drill hole to prevent body fluids from entering the middle ear cavity. Cochlear signals were then amplified (×100 000) and bandpass filtered (0.1 KHz-5 KHz) for subsequent electrophysiological recording. No animal with any evidence of infection or abnormal development was used in the experiment.

Auditory Assessment

The following two auditory recording techniques were used to investigate the potential otoprotective effects of Lamotrigine (LTG):

i) Recording System for Gross Ensemble Spontaneous Activity of Cochlea Nerve Activity (ESAC)

Measurement of the gross ensemble spontaneous activity (ESAC) of cochlea nerve (CN) fibres was carried out by performing a Fast Fourier Transform (FFT) on the time signal generated by the cochlea. This was fed from the amplifier to a PC. The power spectra were then averaged to obtain the average spectrum of CN activity, expressed as μV rms²/Hz. The difference in power between ESAC spectra was calculated in dB [dB=10 log (P1/P2)] where P1 is the pre-treatment FFT and P2 is a post treatment FFT over a given frequency range.

ii) Recording System for Compound Action Potentials (CAP)

The cochlea nerve compound action potential (CAP) was generated in response to discrete acoustic stimuli. It was used to determine both control and treatment related changes in auditory thresholds by measuring changes in CAP latency (milliseconds) and amplitude (μVolts).

Tone ‘pip’ stimuli of 5 millisecond duration were computer generated at 8, 16, 24 and 30 KHz and fed to an attenuator (1 dB resolution). The tone pip was then fed to a Bruel and Kjaer 4192, (microphone) serving as an acoustic driver, which was placed inside an ear speculum coupled to the ear canal. CAP averaging was performed between ×50 to 100 times and the CAP threshold was determined by visual inspection of the recorded time signal. Accuracy was typically between 1-3 dB, using a CAP detection criterion of approximately 1 μV.

### Example 1

### The Assay of Plasma and Perilymph for Lamotrigine (LTG)

In order to establish the otoprotective properties of LTG, a pharmacokinetic evaluation study was designed to establish penetration of LTG into guinea pig perilymph and plasma. A total of 17 healthy adult male albino guinea pigs were used. Plasma and perilymph samples were taken from 3 animals at each timepoint: 1, 2, 5, 10 and 15 hours after dosing. Two untreated control animals were also sampled for comparison.

### Example 1

### Experimental Design

Heparinised blood samples (up to 10 mL) for plasma were taken either via cardiac puncture or exsanguination via the jugular vein and frozen (−20° C.) for subsequent analysis. After the terminal blood sample had been taken, the animal received an overdose of anaesthetic and was decapitated. The bulla was removed and thoroughly cleaned to remove all tissue and fluids. A glass micropipette was then used to puncture the round window membrane and, using gentle aspiration, approximately 6 μL of perilymph was sampled and frozen (−20° C.) for subsequent analysis. Levels of LTG in both plasma and perilymph were determined using a protein precipitation extraction procedure using high performance liquid chromatography (HPLC) with ultraviolet (UV) detection. The LTG retention time was 5.4 minutes and quantification was carried out using peak areas.

### Example 1

### Results

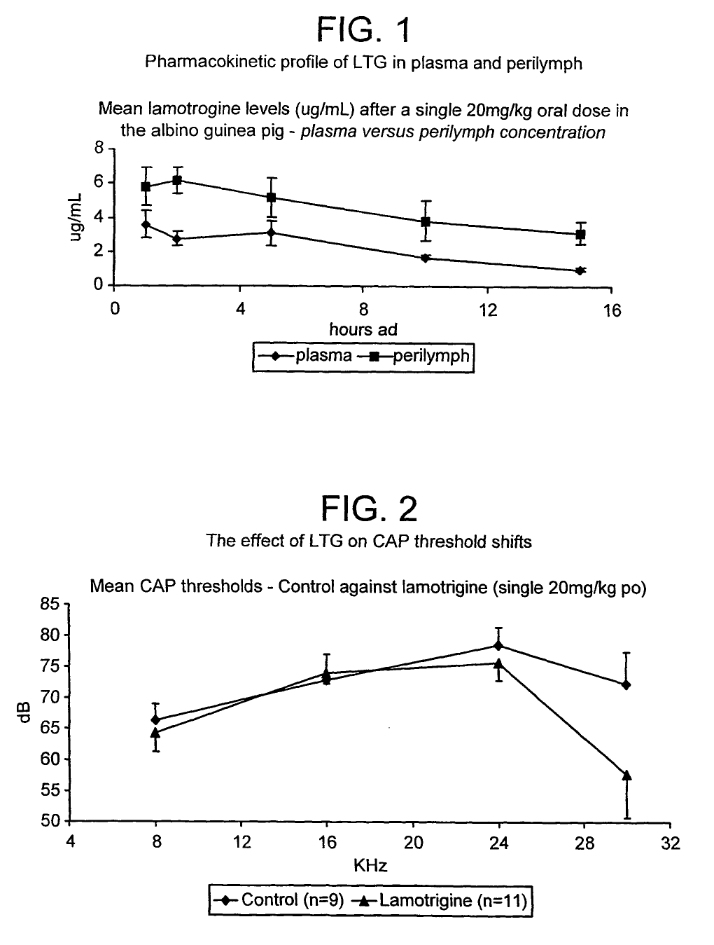

Lamotrigine (LTG) was detected in both plasma and perilymph indicating good systemic exposure and penetration into the inner ear (FIG. 1). Based on the timepoints used in this study, the levels of LTG detected in perilymph were approximately twice those seen in plasma with an observed mean maximum concentration (Cmax) of 6.141 g/mL and an approximate mean time of maximum concentration (Tmax) of 2 hours (mean plasma Cmax value was 3.59 μg/mL with a Tmax of 1 hour). These results show the preferential uptake of LTG by the target organ (the cochlea). Mean plasma and perilymph LTG levels remained relatively high after the 15 hour sampling period (1 and 3.2 μg/mL respectively) indicating comparable elimination rates.

### Example 2

### The Effect of Lamotrigine (LTG) on Normal Auditory Function

### Example 2

### Experimental Design

To determine the effects of LTG on normal auditory function, a total of 20 healthy adult male albino guinea pigs were divided into two groups: Control (n=9) and 20 mg/kg LTG (n=11) administered approximately 2 hours before surgery.

CAP thresholds at 8, 16, 24 and 30 KHz were measured immediately after surgery and again approximately 15 minutes later.

### Example 2

### Results

FIG. 2 shows the effect of LTG on CAP threshold shifts at the four frequencies measured (8, 16, 24 and 30 KHz). There were no statistically significant differences between control and LTG treated animals at any of the 4 frequencies tested.

### Example 3

### Lamotrigine (LTG) Otoprotection After Acute Noise Insult

### Example 3

### Experimental Design

A total of 14 healthy adult male albino guinea pigs were divided into two equal groups: Control (noise alone) and 20 mg/kg LTG (administered approximately 2 hours before noise exposure).

CAP thresholds and ESAC were measured immediately after surgery and again approximately 15 minutes later. Animals were then exposed to 15 minutes filtered (5-20 KHz) free field noise (approximately 10 dB SPL) during which ESAC measurements were made at 5 minute intervals. Post noise CAP threshold and ESAC measurements were taken and the animal was then sacrificed.

### Example 3

### Results

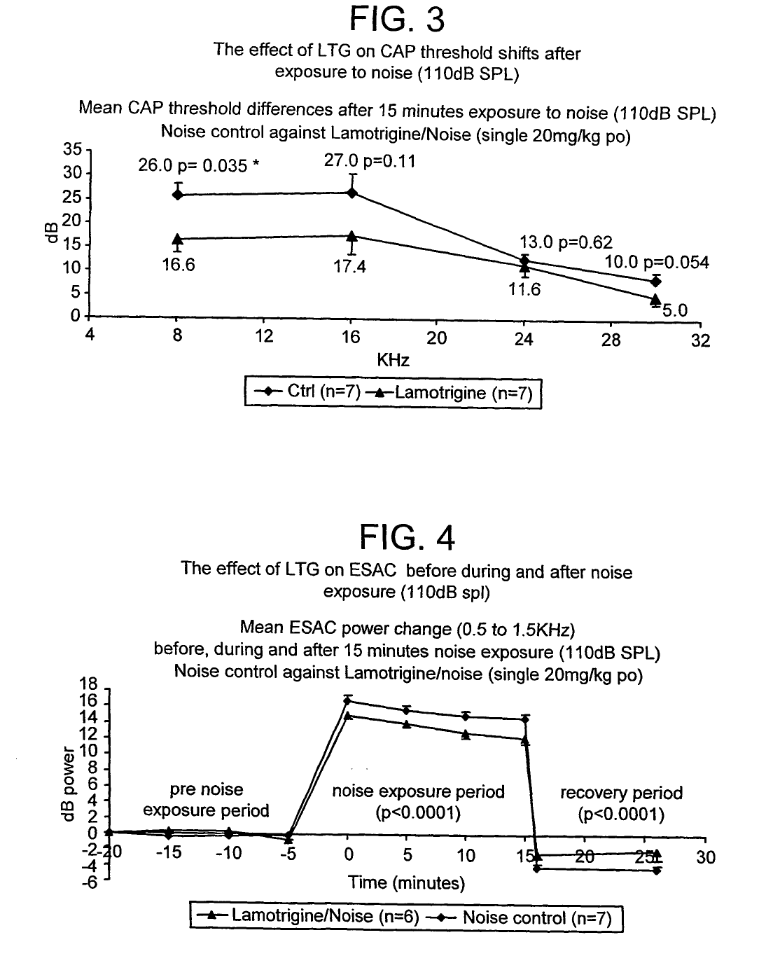

FIG. 3 shows the effect of LTG on CAP threshold shifts at the four frequencies measured (8, 16, 24 and 30 KHz). The appearance of the threshold shift plot reflects the difference of spectral intensity of the filtered noise stimulus (maximum noise stimulus intensity was generated towards 8 KHz to 16 KHz). In all cases mean thresholds were higher in the LTG treated animals and reached statistical significance at 8 kHz (p<0.05).

FIG. 4 shows the effect of LTG on ESAC before during and after noise exposure (only 6 LTG treated animals were used for ESAC measurement). Compared to noise control, a statistically significant decrease (P<0.0001) in the power spectrum of approximately 2 dB was noted in the 20 mg/kg LTG group during noise exposure. This significant decrease continued during the post noise exposure period (p<0.0001). No differences were noted between the two groups prior to noise exposure.

### REFERENCES

Catlin, F. I. (1986). Noise-induced hearing loss [Review]. American Journal of Otology 7, 141-149.

Davis, A. C. (1989). The prevalence of hearing impairment and reported hearing disability among adults in Great Britain. International Journal of Epidemiology (18) 4, 911-917.

Parsons, D. N., Dickins, M and Morley, T. (1995). Lamotrigine: Absorption, distribution and excretion. Antiepileptic drugs, Fourth Edition, Raven Press Ltd., New York.

### Example Pharmaceutical Compositions

### Example 4

### Lamotrigine Pharmaceutical Composition

Lamotrigine tablets for oral administration may formulated with the following ingredients:

| | |
|---|---|
| Lamotrigine | 150 mg |
| Lactose | 200 mg |
| Maize starch | 50 mg |
| Polyvinylpyrrolidone | 4 mg |
| Magnesium stearate | 4 mg |

Mix the active compound with the lactose and starch and granulate with a solution of the polyvinylpyrrolidone in water. Dry the resulting granules, mix with the magnesium stearate and compress to give tablets of average weight 408 mg.

The application of which this description and claims forms part may be used as a basis for priority in respect of any subsequent application. The claims of such subsequent application may be directed to any feature or combination of features described herein. They may take the form of product, composition, process or use claims and may include, by way of example and without limitation, one or more of the following claims:

1. Use of a frequency dependent voltage activated sodium channel blocker or a pharmaceutically acceptable acid addition salt thereof in the manufacture of a medicament for the prevention of noise induced hearing loss.

2. Use according to claim 1 wherein the frequency dependent voltage activated sodium channel blocker is lamotrigine or a pharmaceutically acceptable acid addition salt thereof.

3. A method of preventing noise induced hearing loss in a patient suffering from, or susceptible to, said disorder, which method comprises administering to the patient a therapeutically effective amount of a frequency dependent voltage activated sodium channel blocker or pharmaceutically acceptable acid addition salt thereof.

4. A method according to claim 3 wherein the frequency dependent voltage activated sodium channel blocker is lamotrigine or a pharmaceutically acceptable acid addition salt thereof.

5. A pharmaceutical formulation comprising a frequency dependent voltage activated sodium channel blocker or a pharmaceutically acceptable acid addition salt thereof, together with one or more acceptable carriers or diluents therefor for use in the prevention of noise induced hearing loss.

6. A pharmaceutical formulation according to claim 5 wherein the frequency dependent voltage activated sodium channel blocker is lamotrigine or a pharmaceutically acceptable acid addition salt thereof.
